(19) United States
(12) Patent Application Publication
(10) Pub. No.: US 2021/0154094 A1
(43) Pub. Date: May 27, 2021

(54) MEDICAL DEVICE PACKAGING SYSTEM

(71) Applicant: BOSTON SCIENTIFIC SCIMED, INC., Maple Grove, MN (US)

(72) Inventors: LINDSAY A. SMARON, BOSTON, MA (US); EDWARD A. DZWILL, WESTBOROUGH, MA (US); JAMES M. PECK, JACKSONVILLE, FL (US); AMIT RAUNIYAR, MAPLE GROVE, MN (US)

(73) Assignee: BOSTON SCIENTIFIC SCIMED, INC., MAPLE GROVE, MN (US)

(21) Appl. No.: 17/102,184

(22) Filed: Nov. 23, 2020

Related U.S. Application Data
(60) Provisional application No. 62/940,525, filed on Nov. 26, 2019.

(51) Int. Cl. A61J 1/00; B65D 1/40; B65D 25/10

(52) U.S. Cl. CPC A61J 1/00; B65D 25/103; B65D 1/40

(57) ABSTRACT

Medical device packages are disclosed. An example package for a medical device may include a base portion. The base portion may have a spiral channel formed therein. A cover portion may be coupled to the base portion. The cover portion may be configured to shift between and first position and a second position. The cover portion may overlie the base portion when in the second position. The package may include a securing member for securing the cover portion in the second position.

### CROSS-REFERENCE TO RELATED APPLICATIONS

This application claims the benefit of priority under 35 U.S.C. § 119 to U.S. Provisional Application Ser. No. 62/940,525, filed Nov. 26, 2019, the entirety of which is incorporated herein by reference.

### TECHNICAL FIELD

The present disclosure pertains to medical devices, and methods for manufacturing medical devices. More particularly, the present disclosure pertains to packaging for medical devices.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be packaged in any one of a variety of different packages. Of the known medical devices, methods, and packages, each has certain advantages and disadvantages. There is an ongoing need to provide alternative medical devices as well as alternative methods for manufacturing and using medical devices and alternative packages.

### BRIEF SUMMARY

This disclosure provides design, material, manufacturing method, and use alternatives for medical devices and/or medical device packages. A package for a medical device is disclosed. The package comprises: a base portion, the base portion having a base channel formed therein; a cover portion coupled to the base portion, the cover portion having a cover channel formed therein; wherein the cover portion is configured to shift between and first position and a second position; wherein the cover portion overlies the base portion when in the second position; and a securing member for securing the cover portion in the second position.

Alternatively or additionally to any of the embodiments above, the base channel has a spiral arrangement.

Alternatively or additionally to any of the embodiments above, the cover channel has a spiral arrangement.

Alternatively or additionally to any of the embodiments above, wherein the base portion and the cover portion are a single monolith of material.

Alternatively or additionally to any of the embodiments above, further comprising a hinge region disposed between the base portion and the cover portion.

Alternatively or additionally to any of the embodiments above, the base portion, the cover portion, and the hinge region are a single monolith of material.

Alternatively or additionally to any of the embodiments above, the securing member includes a thermal bond, an adhesive bond, one or more mechanical fittings, or combinations thereof.

Alternatively or additionally to any of the embodiments above, the securing member includes a thermal bond.

Alternatively or additionally to any of the embodiments above, the securing member includes an adhesive.

Alternatively or additionally to any of the embodiments above, the securing member includes one or more mechanical fittings.

Alternatively or additionally to any of the embodiments above, further comprising a patterned surface disposed along the base portion.

Alternatively or additionally to any of the embodiments above, the base portion includes a flushing port.

Alternatively or additionally to any of the embodiments above, further comprising a medical device disposed within the base channel.

A packaging system is disclosed. The packaging system comprises: a clipless coil assembly including an arcuate base member having a spiral channel formed therein; wherein the spiral channel is configured to accommodate a medical device therein.

Alternatively or additionally to any of the embodiments above, the clipless coil assembly is formed of a single monolith of material.

Alternatively or additionally to any of the embodiments above, further comprising a patterned surface disposed along the arcuate base member.

Alternatively or additionally to any of the embodiments above, further comprising the medical device disposed within the spiral channel.

A packaging system is disclosed. The packaging system comprises: a base including a medical device holding channel, the medical device holding channel having a spiral arrangement; a cover integrally formed with the base; and wherein the cover is configured to shift between an open configuration and a closed configuration where the cover overlies the base.

Alternatively or additionally to any of the embodiments above, the base includes a spiral ridge and wherein the medical device holding channel is formed along the spiral ridge.

Alternatively or additionally to any of the embodiments above, the cover includes a spiral ridge and wherein a cover channel is formed along the spiral ridge.

Alternatively or additionally to any of the embodiments above, further comprising a patterned surface disposed along the base.

Alternatively or additionally to any of the embodiments above, further comprising a securing member for securing the cover in the closed configuration.

Alternatively or additionally to any of the embodiments above, further comprising a medical device disposed within the medical device holding channel.

A package for a medical device is disclosed. The package comprises: a base portion; a cover portion coupled to the base portion, the cover portion being configured to shift between and first position and a second position; wherein the cover portion includes a deformable membrane; wherein the cover portion overlies the base portion when in the second position; a securing member for securing the cover portion in the second position; and a hinge region disposed between the base portion and the cover portion.

Alternatively or additionally to any of the embodiments above, the base portion has a spiral channel formed therein.

Alternatively or additionally to any of the embodiments above, wherein the deformable membrane includes silicone.

A package for a medical device is disclosed. The package comprises: a base portion, the base portion having a base channel formed therein; a cover portion coupled to the base portion, the cover portion having a cover channel formed therein; wherein the cover portion is configured to shift between and first position and a second position; wherein the cover portion overlies the base portion when in the second position; a securing member for securing the cover portion in the second position; and a hinge region disposed between the base portion and the cover portion.

A packaging system is disclosed. The packaging system comprises: a base including a medical device holding channel, the medical device holding channel having a spiral arrangement; a cover integrally formed with the base; wherein the cover is configured to shift between an open configuration and a closed configuration where the cover overlies the base; and a securing member for securing the cover in the closed configuration.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a top view of an example medical device package.

FIG. 2 is a cross-sectional view of a portion of an example medical device package.

FIG. 3 is a cross-sectional view of a portion of an example medical device package.

FIG. 4 is a cross-sectional view of a portion of an example medical device package.

FIG. 5 is a top view of an example medical device package.

FIG. 6 is a top view of an example medical device package.

FIG. 7 is a cross-sectional view of a portion of an example medical device package.

FIG. 8 is a cross-sectional view of a portion of an example medical device package.

FIG. 9 depicts a portion of an example medical device package.

FIG. 10 depicts a portion of an example medical device package.

FIG. 11 depicts a portion of an example medical device package.

FIG. 12 is a top view of an example medical device package.

FIG. 13 is a top view of an example medical device package.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Medical devices are packaged and stored within a package, packaging system, packaging assembly, and/or the like. Some devices like catheter and guidewire are typically loaded into a carrier tube. The carrier tube may be arranged in a coiled/spiral configuration where adjacent windings of the carrier tube are held together with a clip. In some instances, a flush port or fitting (e.g., a luer fitting) may be disposed at an end of the carrier tube so as to help facilitate flushing of the medical device. Disclosed herein are alternative packages that include a number of desirable features.

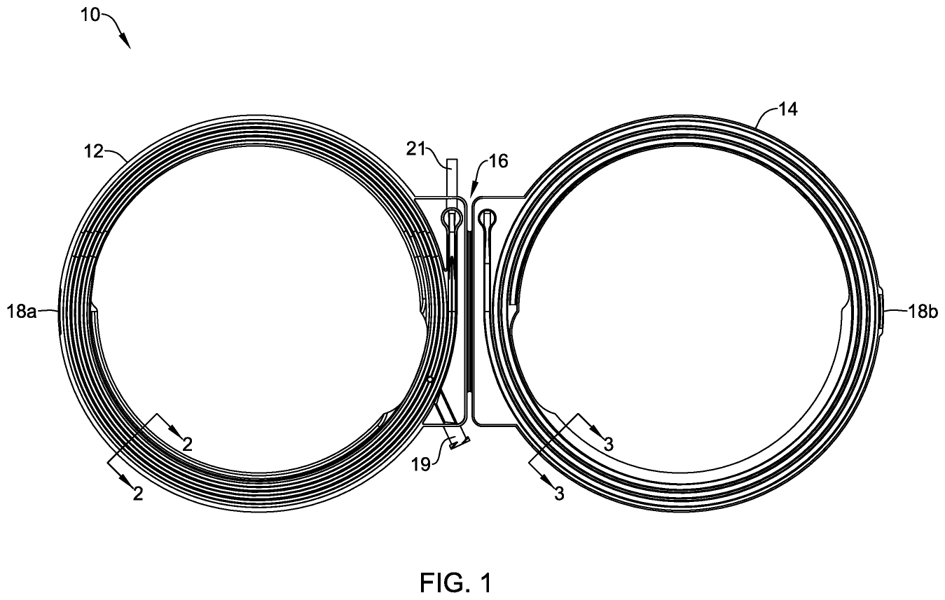

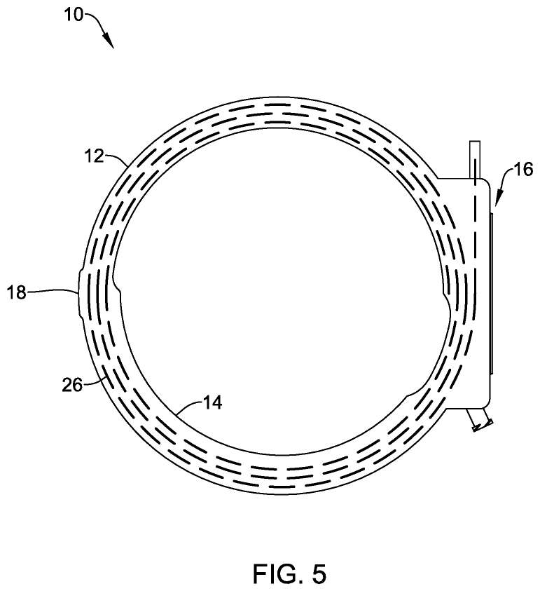

FIG. 1 is a top view of an example package 10. The package 10 may include a first or base portion 12 and a second or cover portion 14. The base portion 12 and the cover portion 14 may be coupled to one another by or at a hinge region 16. As such, the cover portion 14 may be designed to shift between a first or open configuration (e.g., as depicted in FIG. 1) and a second or closed configuration (e.g., as depicted in FIG. 5). The package 10 may have a clamshell configuration where the cover portion 14 overlies and covers the base portion 12 when in a closed configured.

In some instances, the base portion 12 and the cover portion 14 are integral with one another. In other words, the base portion 12 and the cover portion 14 may be formed from a singular monolith of material. In some instances, the base portion 12, the cover portion 14, and the hinge region 16 may be formed from a single monolith of material. Alternatively, one or more of the base portion 12, the cover portion 14, and/or the hinge region 16 may be formed as a separate component and then the separate component(s) are secured to one another.

In some instances, the base portion 12, the cover portion 14, or both may include one or more structural features generally configured to secure the cover portion 14 to the base portion 12. For example, the base portion 12 may include one or more securing members 18a and the cover portion 14 may include one or more securing members 18b configured to engage the securing members 18a of the base portion 12. In FIG. 1, the securing member(s) 18a, 18b are depicted at mechanical fittings (e.g., snap fittings) that are designed to engage/interlock with one another. However, this is not intended to be limiting. For example, the securing member(s) (e.g., the securing member 18a, 18b, or both) may take the form of an adhesive bond, a thermal bond, a weld (e.g., an ultrasonic weld), or the like. Still other securing mechanisms are contemplated including slide and lock mechanisms, twist/thread securing mechanisms, etc.

In at least some instances, the package 10 may be formed by a suitable process such as molding, injection molding, thermoforming, or the like. This may include forming the package 10 to include structures resembling some of the features of other medical device packages. Unlike other medical device packages, however, the package 10 allows for the packaging of a medical device (e.g., such as a guidewire, catheter, stent delivery system, or the like) without the need for a carrier tube, without the need of clips holding together adjacent windings of a carrier tube, and with structures that allow for relatively simple flushing of the medical device. In some instances, the package 10 may be described as being a carrier-tube free package 10, a clipless package 10, or the like. By omitting these additional structures, package manufacturing costs, assembly labor, coordination of differently sized components, and the like can be reduced. Furthermore, because a medical device may simply lie within a channel formed in the base portion 12 and/or the cover portion 14, the medical device may be flushed by simply placing the base portion 12 in a suitable bath, transporting the medical device directly to the suitable bath, flushing the medical device with a syringe or suitable device, combinations thereof, and/or the like. In some instances, the package 10 may include one or more flush ports 19, 21 that may help to facilitate flushing. In addition, the design of the package 10 may help to keep the overall footprint of the packaging to be reasonably small.

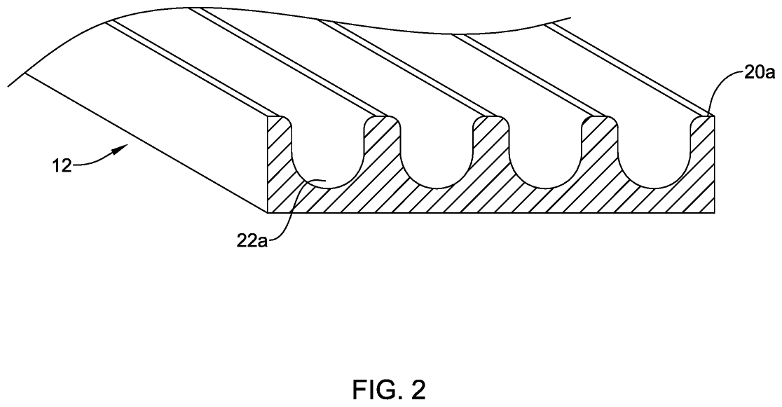

FIG. 2 is a cross-sectional view of a portion of an example medical device package 10 taken along line 2-2 of FIG. 1. In order to accommodate a medical device therein, the package 10 may include structural features designed to hold a medical device. For example, the base portion 12 may include a ridge 20a (e.g. one or more ridges 20a) defining a channel 22a (e.g., one or more channels 22a) along the base portion 12 as shown in FIG. 2. In some instances, the ridge 20a may project from the base of the base portion 12 and may be arranged in a spiral arrangement. Other arrangements are contemplated including linear arrangements, arcuate or curved arrangements, helical arrangements, irregular arrangements, arrangements that form a geometric shape (e.g., a triangular arrangement, a square arrangement, a rectangular arrangement, etc.), and/or the like. A spiral arrangement of the ridge 20a may result in a spirally-arranged channel 22a. The spirally-arranged channel 22a may allow for the medical device disposed therein to be held in a generally atraumatic spiral arrangement. In some instances, the channel 22a is merely defined between adjacent windings of the ridge 20a and the medical device may simply lie within the channel 22a. Such as design allows the medical device to easily be disposed within and removed from the channel 22a while subjecting the medical device to minimal loading/removal forces. In addition, the channel 22a may reduce the likelihood of frictional forces being present between the medical device and the package 10.

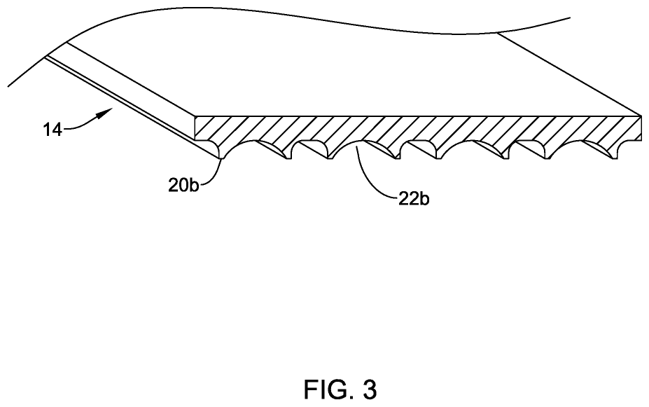

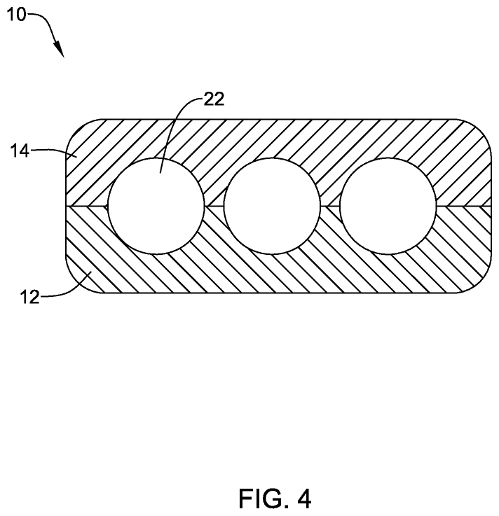

FIG. 3 is a cross-sectional view of a portion of an example medical device package 10 taken along line 3-3 of FIG. 1. The cover portion 14 may also include a ridge 20b (e.g. one or more ridges 20b) defining a channel 22b (e.g., one or more channels 22b) along the cover portion 14 as shown in FIG. 3. In some instances, the ridge 20b may project from the base of the cover portion 14 and may be arranged in a spiral arrangement. In general, the arrangement of the ridge 20b and/or the channel 22b may be designed so that when the cover portion 14 is brought together with the base portion 12, they form a channel 22 through the package 10 as shown in FIG. 4. The channel 22 can accommodate a medical device 26 as schematically depicted in FIG. 5. The medical device 26 may include a guidewire, catheter, balloon catheter, stent or implant delivery system, or the like.

Alternative cover portions 14 are contemplated that may lack a ridge 20b and/or a channel 22b. In these instances, the medical device 26 may be disposed along the base portion 12 and be held within the channel 22a. In these instances, when the cover portion 14 overlies the base portion 12, the cover portion 14 (e.g., which may include a generally planar surface that covers the base portion 12) may entrap the medical device 26 within the channel 22a. In other instances, the base portion 12 may lack a ridge 20a and/or a channel 22a. In these instances, the medical device 26 may be disposed along the base portion 12 and be held within the channel 22b when the cover portion 14 overlies the base portion 12.

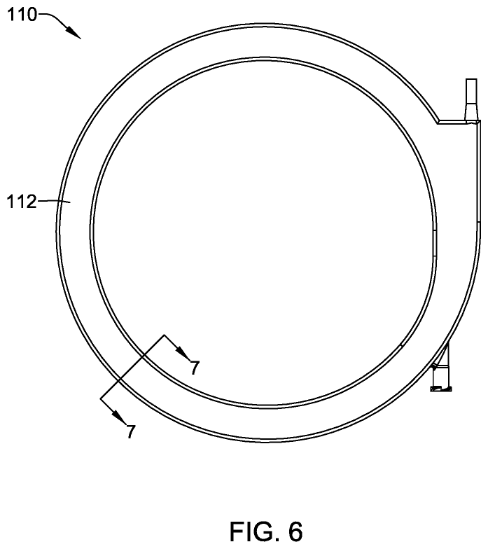

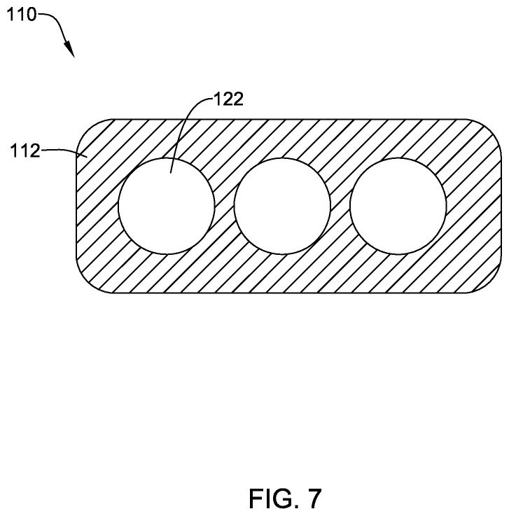

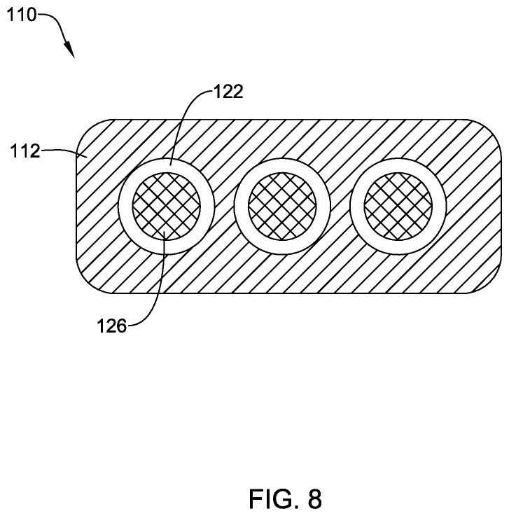

While FIGS. 1-5 depict a package 10 with a clamshell configuration, other packages are contemplated. For example, FIGS. 6-8 illustrates another example package 110 that may be similar in form and function to other packages disclosed herein. In this example, rather than being two mating halves of a clamshell container, the package 110 includes a unitary arcuate base portion 112 with a channel 122 (e.g., a spiral channel 122) formed therein, as shown in FIG. 7 which is cross-section taken along line 7-7 of FIG. 6. Such a structure may resemble a carrier tube arranged in a spiral arraignment but, because the base portion 112 is a unitary structure, the package 110 does not include clips holding together adjacent windings of a carrier tube. In other words, the package 110 may be described as being “clipless” or as resembling or being a “clipless coil assembly”. The channel 122 may allow a medical device 126 to be accommodated therein as schematically depicted in FIG. 8.

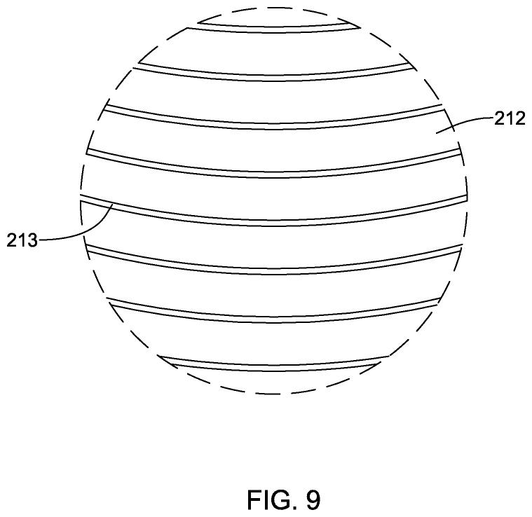

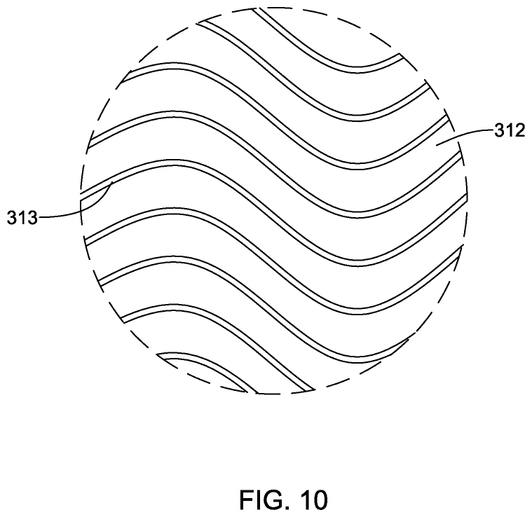

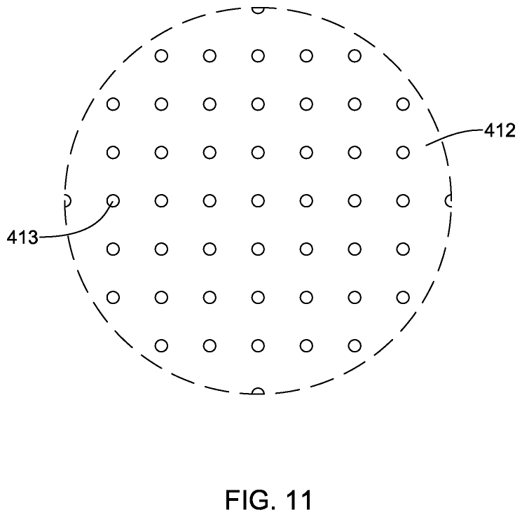

In some instances, the base portion 12, 112 may include a structured or pattered surface. The patterned surface may be disposed along essentially the entire base portion 12, 112 (e.g., along the full “top” surface), along sections of the base portion 12, 112, along the channel 22, 122, combinations thereof, or the like. For the purposes of this disclosure, the patterned surface need not include a regular pattern formed in the base portion 12, 112 as patterns that are regular, irregular, random, and the like are contemplated. The patterned surface may help to reduce contact between a medical device disposed along the base portion 12, 112 (e.g., within the channel 22, 122). For example, this may allow a device to be easily inserted/removed from the package 10, 110. Example patterned surfaces are depicted in FIGS. 3-5. For example, FIG. 9 depicts a base portion 212 with a patterned surface 213. In this example, the patterned surface 213 takes the form of a spiral groove or concentric arcuate slots/grooves. It can be appreciated that patterned surfaces are contemplated that are similar to the patterned surface 213, but project from the base portion 212 instead of being slots/grooves formed in the base portion 212. FIG. 10 depicts another example base portion 312 with a patterned surface 313. In this example, the patterned surface 313 includes a number of wavy or wave-like projections. It can be appreciated that patterned surfaces are contemplated that are similar to the patterned surface 313, but take the form of grooves/slots formed in the base portion 312 instead of being projections. FIG. 11 depicts another example base portion 412 with a patterned surface 413. In this example, the patterned surface 413 takes the form of a plurality of dimples or indentations. It can be appreciated that patterned surfaces are contemplated that are similar to the patterned surface 413, but project from the base portion 412 instead of being indentations formed in the base portion 412. The patterned surfaces 213, 313, 413 may be formed by a suitable process. For example, the patterned surfaces 213, 313, 413 as part of molding process, by micro-machining or micro-sculpting, by ablation, by mechanical scoring, and/or the like.

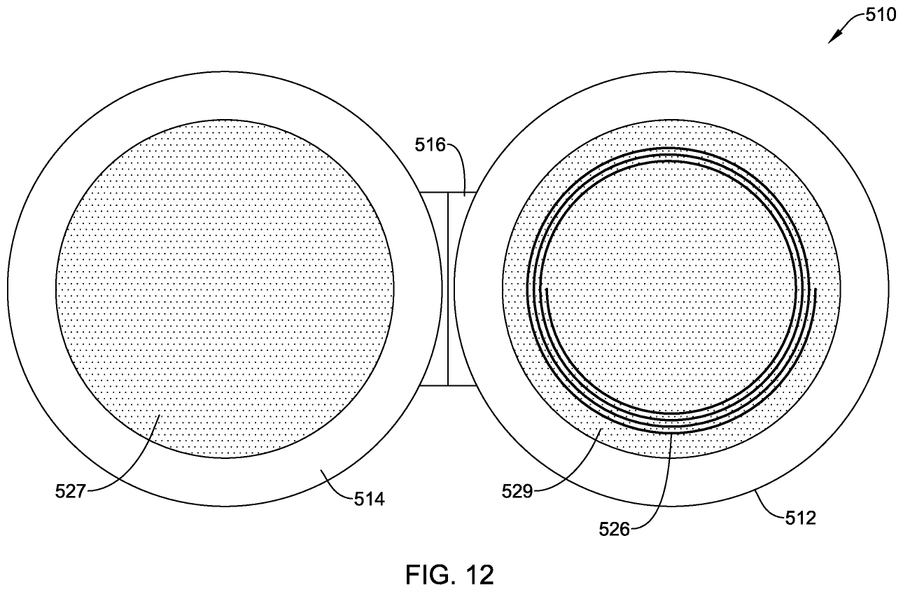

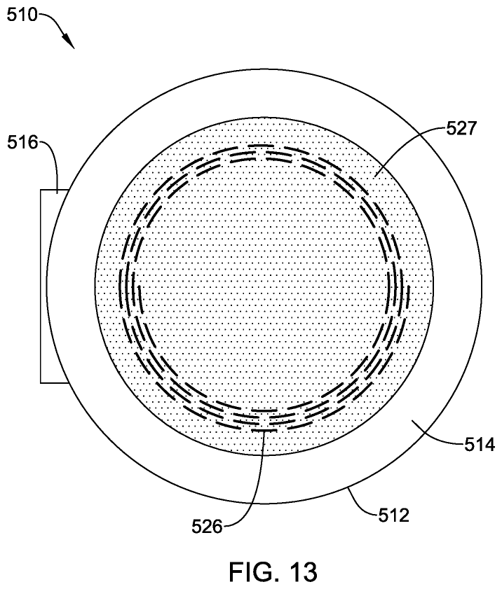

FIGS. 12-13 illustrate another example package 510 that may be similar in form and function to other packages disclosed herein. The package 510 may include a base portion 512, a cover portion 514, and a hinge 516. In some instances, the base portion 512 may include a medical device receiving region 529. The medical device receiving region 529 may be a relatively rigid material or the medical device receiving region 529 may include a deformable material such as silicone. In some instances, the perimeter of the base portion 512 may be formed from a relatively rigid material and the medical device receiving region 529 may include a deformable material such as silicone. The cover portion 512 may include a region 527. In some instances, the region 527 may include a deformable material such as silicone. In some instances, the perimeter of the cover portion 514 may be formed from a relatively rigid material and the region 527 may include a deformable material such as silicone. A medical device 526 may be disposed along the medical device receiving region 529. When the cover portion 514 is closed to overlie the base portion 512 as depicted in FIG. 13, the medical device 526 may be secured between the base portion 512 and the cover portion 514. In instances where the region 527 includes a deformable material, the deformable material may collapse onto the medical device 526. In instances where both the medical device receiving region 529 and the region 527 of the cover portion 514 includes a deformable material, the deformable materials may collapse onto the medical device 526.

In some instances, any of the packages/packaging systems disclosed herein may allow for sterilization processes typically used for medical devices such e-beam sterilization, ethylene oxide sterilization, etc. In some instances, the packages may be disposed within a secondary container or pouch (not shown) and sterilized. In some instances, the secondary container may include one or more walls or regions with a breathable material (e.g., such as high density polyethylene fibers such as TYVEK). In other instances, the secondary container may not be necessary and can be omitted. In such instances, the medical device can be sufficiently sterilized (e.g., and kept sterile) by securing/sealing the medical device within the package.

The medical device packages disclosed herein may be formed from or otherwise includes suitable materials such as polymers. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A package for a medical device, the package comprising:
a base portion, the base portion having a base channel formed therein;
a cover portion coupled to the base portion, the cover portion having a cover channel formed therein;
wherein the cover portion is configured to shift between and first position and a second position;
wherein the cover portion overlies the base portion when in the second position; and
a securing member for securing the cover portion in the second position.

2. The package of claim 1, wherein the base channel has a spiral arrangement.

3. The package of claim 1, wherein the cover channel has a spiral arrangement.

4. The package of claim 1, wherein the base portion and the cover portion are a single monolith of material.

5. The package of claim 1, further comprising a hinge region disposed between the base portion and the cover portion

6. The package of claim 5, wherein the base portion, the cover portion, and the hinge region are a single monolith of material.

7. The package of claim 1, wherein the securing member includes a thermal bond, an adhesive bond, one or more mechanical fittings, or combinations thereof.

8. The package of claim 1, further comprising a patterned surface disposed along the base portion.

9. The package of claim 1, wherein the base portion includes a flushing port.

10. The package of claim 1, further comprising a medical device disposed within the base channel.

11. A packaging system, comprising:
a clipless coil assembly including an arcuate base member having a spiral channel formed therein;
wherein the spiral channel is configured to accommodate a medical device therein.

12. The packaging system of claim 11, wherein the clipless coil assembly is formed of a single monolith of material.

13. The packaging system of claim 11, further comprising a patterned surface disposed along the arcuate base member.

14. The packaging system of claim 11, further comprising the medical device disposed within the spiral channel.

15. A packaging system, comprising:
a base including a medical device holding channel, the medical device holding channel having a spiral arrangement;
a cover integrally formed with the base; and
wherein the cover is configured to shift between an open configuration and a closed configuration where the cover overlies the base.

16. The packaging system of claim 15, wherein the base includes a spiral ridge and wherein the medical device holding channel is formed along the spiral ridge.

17. The packaging system of claim 15, wherein the cover includes a spiral ridge and wherein a cover channel is formed along the spiral ridge.

18. The packaging system of claim 15, further comprising a patterned surface disposed along the base.

19. The packaging system of claim 15, further comprising a securing member for securing the cover in the closed configuration.

20. The packaging system of claim 15, further comprising a medical device disposed within the medical device holding channel.
